(19) United States
(12) Patent Application Publication
(10) Pub. No.: US 2015/0111787 A1
(43) Pub. Date: Apr. 23, 2015

(54) DIGITAL MICROFLUIDIC CHIPS FOR AUTOMATED HYDROGEN DEUTERIUM EXCHANGE (HDX) MS ANALYSIS

(71) Applicants: Kwang-Fu C. Shen, Westlake Village, CA (US); Julian P. Whitelegge, Costa Mesa, CA (US); Kym Francis Faull, Los Angeles, CA (US); Libo Zhao, Los Angeles, CA (US); Christopher Miles Ryan, Los Angeles, CA (US)

(72) Inventors: Kwang-Fu C. Shen, Westlake Village, CA (US); Julian P. Whitelegge, Costa Mesa, CA (US); Kym Francis Faull, Los Angeles, CA (US); Libo Zhao, Los Angeles, CA (US); Christopher Miles Ryan, Los Angeles, CA (US)

(73) Assignee: THE REGENTS OF THE UNIVERSITY OF CALIFORNIA, Oakland, CA (US)

(21) Appl. No.: 14/123,502

(22) PCT Filed: Jun. 4, 2012
(86) PCT No.: PCT/US2012/040792

§ 371 Date: Nov. 18, 2014

Related U.S. Application Data
(60) Provisional application No. 61/492,750, filed on Jun. 2, 2011.

(51) Int. Cl. G01N 33/68; G01N 35/10; G01N 30/88; B01L 3/00

(52) U.S. Cl. CPC G01N 33/6842; B01L 3/502784; G01N 33/6848; G01N 35/1095; G01N 30/88; B01L 2300/0867; B01L 2200/0673; B01L 2200/027; G01N 2458/15; G01N 2030/8831
USPC 506/12; 506/37

(57) ABSTRACT

Described herein is a digital microfluidic droplet generator (DMDG) and a microfluidic platform for processing material introduced into the DMDG. The combination is particularly suited for hydrogen/deuterium exchange and mass spectrometer (HDX-MS) processing and analysis of membrane proteins.

Benefit is claimed of Provisional Patent Application Ser. No. 61/492,750, filed Jun. 2, 2011.

### FIELD OF INVENTION

The following disclosure relates to the field of protein analysis, and more specifically, membrane protein analysis, using digital microfluidic droplet generators and automated microfluidic platforms for processing, reacting, identifying and analyzing protein structures. In particular HDX-MS procedures using microfluidic chips are addressed.

### BACKGROUND OF THE INVENTION

Mass-based hydrogen/deuterium exchange (HDX-MS) procedures probe protein structures by monitoring the rate and extent of deuterium exchange with backbone amide protons. Hydrogen/deuterium exchange (HDX) is one of the most powerful and versatile techniques for probing the conformational dynamics of proteins in solution. Over the past quarter century, HDX studies have yielded valuable insights into protein dynamics, protein folding and protein-ligand interactions. HDX measured using mass spectrometry (HDX-MS) has also emerged as an important tool for structure biologists. HDX-MS exploits the fact that backbone amide hydrogens can exchange with deuterium when a protein is incubated in D₂O, and that the rate of the exchange process is highly dependent on the local structural environment. Features of HDX-MS that make it an especially attractive approach include small sample requirements and the ability to study extremely large protein assemblies that are not amenable to other techniques.

This approach has proven to be a useful method for documenting protein dynamics and changes to protein conformation in solution. (Faull, K. F., Higginson, J., Waring, A. J., To, T., Whitelegge, J. P., Stevens, R. L., Fluharty, C. B., Fluharty, A. L. J. Mass Spectrometry, 2000, 35, 392; Konermann, L., Pan, J., Yu, Y.-H. Chem. Soc. Rev. 2010, 40, 1244). Integral proteins of lipd bilayer membranes represent one third of the cellular proteome and include many potential therapeutic targets. However, analyzing membrane proteins using HDX-MS is challenging due to the hydrophobic transmembrane domains that limit the solubility of these proteins in aqueous solvents. In addition, the HDX process is sensitive to environmental factors including, but not limited to, temperature, pH, salt concentration, and detergents used for solubilization, as well as operation duration and processing sequences. As a result, low reproducibility of experimental results are often observed. Therefore, technical solutions are desired that allow improved handling of integral membrane proteins and help the analysis of transmembrane domains.

Lars Konermann et al. have applied stopped-flow and continuous flow microfluidic systems for MS-based kinetic studies of protein folding in the millisecond regime. They also used similar systems for HDX experiments. However, in their publication (J. Pan, J. Han, C. H. Borchers, and L. Konermann, “Hydrogen/Deuterium Exchange Mass Spectrometry with Top-Down Electron Capture Dissociation for Characterizing Structural Transitions of a 17 kDa Protein” J. Am. Chem. Soc., 131, p. 12801-12808 (2009)), they still utilized two syringe pumps using a tee junction for on-line mixing of protein and D₂O and then mixed in the “quencher” (0.4% formic acid in CH₃CN/H₂O/D₂O=1:1.8:7.2) before injecting into an ESI-MS for top-down electron capture dissociation using HDX MS. In addition, the model protein used in their study was myoglobin, which is not a membrane protein and as such is very soluble in buffered aqueous solutions. Thus, they had no need to implement a chromatographic step in-between for separation of proteins.

Further utilization of top-down proteomic strategies requires advances in critical technical areas, including throughput and sensitivity, and coverage of diverse classes of proteins that currently require protein-specific approaches for effective analytical protein chemistry. However, membrane proteins are typically poorly represented in current top-down workflows, especially for mammalian systems, due to their extremely amphipathic physical characteristics. Set forth herein are refinements of various protocols that enable and empower top-down high-resolution mass spectrometry of integral membrane protein primary structure including post-translational modifications.

### SUMMARY

Described herein is a digital microfluidic droplet generator (DMDG) and a microfluidic platform for processing material introduced into the DMDG. The combination is particularly suited for hydrogen/deuterium exchange and mass spectrometer (HDX-MS) processing and analysis of membrane proteins.

### BRIEF DESCRIPTION OF DRAWINGS

FIG. 1 is a schematic representation of a DMDG chip and chromatography setup for HDX-MS.

FIG. 2 is a chart showing the general process flow for mass spectrometry-based assays.

FIG. 3 is a photograph illustrating a two-layer PDMS microfluidic droplet generator chip.

FIGS. 4a-4e show various components of FIG. 3. The different shades of gray coloring in FIGS. 3 and 4a-e represent colored dyes in the channels use for illustration purposes.

FIG. 5 is a graph showing the results of chip HDX-MS analysis of myoglobin (Mb).

FIG. 6 is a graph showing on chip HDX-MS analysis of bacteriorhodopsi (BR).

### DETAILED DESCRIPTION

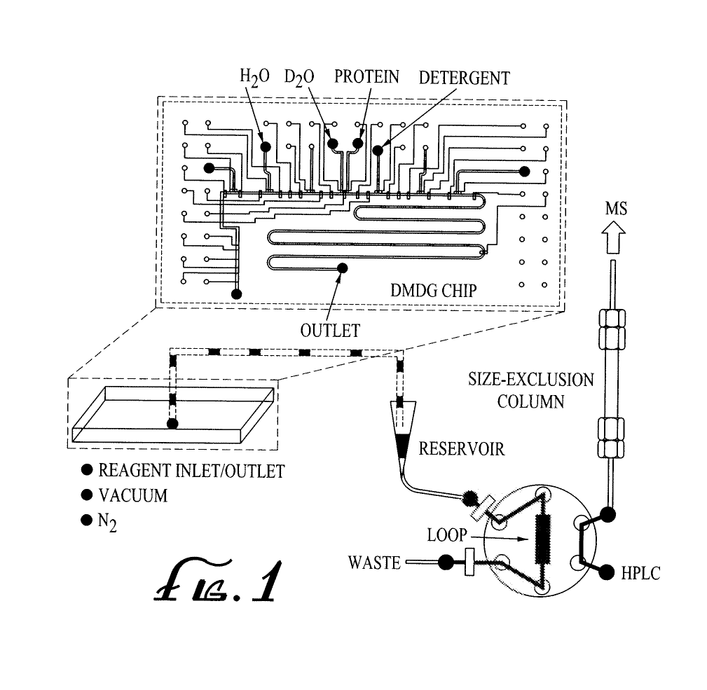

The Digital Microfluidic Droplet Generator (DMDG) Chip (K. Liu, K., Lepin, E. J., Wang, M.-W., Guo, F., Lin, W.-Y., Chen, Y.-C., Olma, S., Phelps, M. E., Zhao, X.-Z., Tseng, H. R., van Dam, R. M., Wu, A. M., Shen, C. K.-F. Molecular Imaging, 2010, 10, 168; Liu, K., Chen, Y. C., Tseng, H. R., Shen, C. K.-F., van Dam, R. M. van Dam. Microfluid Nanofluid, 2010, 9, 933; Wang, H., Liu, K., Chen, K.-J., Lu, Y., Wang, S., Guo, F., Lin, W.-Y., Kamei, K.-I., Chen, Y.-C., Ohashi, M., Wang, M., Zhao, X.-Z., Shen, C. K.-F., Tseng, H.-R. ACS Nano. 2010, 4(10), 6235.) is an automated microfluidic platform that generates nanoliter droplets with well-defined compositions preprogrammed by the user (FIG. 1). Since only small amounts of a sample are required, the DMDG has been used to identify optimal radiolabeling conditions of proteins and peptides, and to screen for the optimal combination of individual building blocks for assembling supramolecular nanoparticles (SNPs) with tailored biological properties. Shown and described herein are improvements to the application of the DMDG device including the results of successful multi-step use of HDX. One of the biggest problems using HDX-MS to probe protein structures is the back-exchange (from D ((deuterium)) back to H ((hydrogen)) after the initial HDX has been quenched). Due to different degrees of back-exchange pertinent data can be lost and data analysis can be complicated and confusing.

Described herein are new techniques for the application of digital microfluidic chip technology such as shown in FIG. 1 in the field of protein structure analysis, particularly a structural analysis of membrane proteins.

Integral membrane proteins present many technical challenges for efficient mass spectrometry in a large part due to hydrophobic transmembrane domains that greatly limit their solubility in aqueous solvents. Prior techniques for the use of HDX-MS have several deficiencies when used to analyze membrane proteins. One reason is the low reproducibility of experiment results because the HDX process is very sensitive to various environmental difference (temperature, pH, salt concentration, detergents, etc.) and operational sequencing. In addition, a significant problem with solution-phase HDX as a mass spectrometric probe of surface exposure in a protein or protein complex is the back-exchange, for example H for D after the initial H/D exchange has been quenched. Different levels of back-exchange result in loss of pertinent data as well as greatly hampering data analysis. In past attempts to minimize these problems, very fast HPLC was performed at low temperature (0-4° C.) to help reduce rate of back-exchange. However, the calculated back-exchange still averages about 30%. New techniques described herein improve the capability to handle integral membrane proteins and analyze transmembrane domains.

Generally, a mass spectrometry-based assay is composed of five steps as shown in FIG. 2:
(1) mixing reagent(s) with samples;
(2) incubation;
(3) post-experiment processing;
(4) separation (i.e., by chromatography) and
(5) analysis by MS.

In order to accomplish this procedure in the most efficient manner and maintain the integrity of the assay results, an automated platform, which can manipulate small amounts of a samples and to perform HDX very rapidly and interfaced with HPLC-ESI-MS systems, such as shown in FIG. 1, is highly desirable. The digital microfluidic droplet generator (DMDG) chip described herein can be readily interfaced with any type of mass spectrometers. The DMDG, is an ideal platform to meet all the requirements for an ESI-MS-based biological assay for routine process use. Described herein is the digital microfluidic droplet generator (DMDG) chip, shown herein, using on-chip HDX and subsequent high-throughput microfluidics/mass spectrometry adapted for evaluation, analysis and screening assays of small molecules that modulate membrane protein stability.

The rates of backbone amide hydrogen/deuterium exchange (HDX) in solution can provide a wealth of information about the structures and conformational dynamics of proteins and protein complexes. For example, a substantial reduction of H/D exchange rates of different amide sites can be observed as a result of either intramolecular hydrogen bonding or sequestration of the amide hydrogen atoms from bulk solvent. Thus, these exchange rates are useful probes of higher-order structure and thermodynamics of local or global unfolding events. Using MS to detect such HDX is very useful to characterize individual conformational states of a protein that may coexist in equilibrium in the solution. Because of its superior sensitivity, MS requires only minute amounts of proteins, for example in the sub-femtomolar range, for analysis, thus allowing proteins to be studied at endogenous levels, which is a great advantage when compared to other techniques such as NMR spectroscopy. Shown herein are front-end HDX operations automated in a microfluidic platform. By combining these two analytical methods, microfluidic-based HDX-MS technology is demonstrated herein to provide a unique advantage in the characterization of protein drugs, visualization of protein ligand interaction in real-time solution-phase protein structure determination and screening ligands that can stabilize structures of membrane proteins

Characterization of protein drugs—One of the major challenges in characterizing protein therapeutics is to define their higher order structures and the conformational dynamics that often dictate their biological activity, stability and safety such as protein conformational changes upon modifications, non-covalent interactions between small molecules and receptor proteins, and protein aggregation caused by misfolding. Currently it is difficult to measure their conformation in solution and in a real-time fashion because the presence of biological matrices and complex serum/plasma proteins from in vivo samples often requires careful sample purification and/or preparation before analysis. Furthermore, arrays of protein therapeutics including, but not limited to cytokines, enzymes, peptides, monoclonal antibodies and fragments present great complexity in sample analysis and characterization. The quantitative measurements by HDX-MS assay is an orthogonal approach to traditional immunoassays. Preliminary studies demonstrate the reliability, robustness and sensitivity of these techniques. The microfluidic-based HDX-MS assay techniques describe herein have been found to be a suitable alternative for these evaluations in routine QC process of given protein therapeutics.

Visualization of protein ligand interaction in real-time—Protein function is highly correlated with its structure. Drugs, hormones and other binding molecules (e.g. peptides or other proteins) modulate target protein's activity by causing structural changes that will produce activation or inactivation of cellular processes. The ability to map new interaction sites and detect novel conformational changes are of great importance when developing small molecule- or antibodies-based drugs targeting certain proteins. However, one of the major limitations in developing drugs is the lack of rapid and reliable analytical methods that allow real-time monitoring of protein-ligand interactions. This is due, to a large extent, to the transient nature of the interactions between proteins and their binding partners. The solution- based mass spectrometry (MS) analyses, including HDX-MS, are tools suitable for addressing this issue and are used to detect the presence of a protein-ligand interaction and to determine its dissociation constant K_{d}. However, in pharmaceutical research, there is an increasing need for screening interactions between proteins and ligands in a high-throughput and reliable matter. Such tasks, which are not being suitably accomplished with prior art techniques, are readily accomplished with the microfluidic HDX-MS approach described herein.

Solution-phase protein structure determination—Well-established techniques for protein structure determination, such as X-ray crystallography and NMR, require substantial quantities of material to be tested and are not particularly suitable for higher molecular weight and dynamic of proteins. On the other hand, HDX-MS analysis measures global or local change in protein conformation using much smaller amounts of material and is suitable for a wide range of proteins. For example, HDX-MS analysis can be used to measure changes in protein conformation. When bottom-up HDX-MS is conducted in combination with HPLC separations and high-resolution MS (along with NMR and/or X-ray crystallography), it is possible to determine the protein structure and discover subtle changes in conformation. Microfluidic-based HDX MS assay can also create samples with various degree of deuteration and/or denaturation for systematic structural analyses.

Screening ligands that can stabilize structures of membrane proteins—The greatest difficulty in working with membrane proteins is their insolubility in water and their lack of stability in detergent-solubilized form. Low stability leads to heterogeneity and aggregation which can preclude or retard the ability to determine the protein structure by current techniques. Current high-throughput (HT) approaches for stability screening are not generally applicable to membrane proteins, particularly in the presence of detergents. While techniques exist for monitoring membrane protein folding, they are not easy to perform and are not amenable to HT approaches. The microfluidic HDX-MS platform described herein addresses these problems and therefore can be used for screening libraries of small molecules that can potentially stabilize the membrane protein of interest and maintain its structural integrity during crystallization.

The low-sample consumption and high-throughput capability of digital microfluidics to facilitate screening processes, as set forth herein, significantly improve and accelerate the downstream workflow of each application. This versatile platform can also be very useful for investigation of drug/ligand-protein interaction, binding pocket mapping and the study of protein folding dynamics. As a result, pharmaceutical and biotech companies can use this approach to streamline their drug discovery and R&D development as well as providing quality control of their protein-based therapeutics.

No existing publication or literature shows or suggests the concept of performing the entire HDX procedure on a specifically designed chip in an automated fashion as well as coupling that procedure with fast chromatographic separation at room temperature and MS analysis.

The microfluidic-assisted HDX techniques described herein provide several advantages over other technologies or platforms:

(1) Sample economy—Microfluidics can handle and manipulate micro- to nanoliter quantities of liquid easily and thus reduce the loss of processed materials. The very small quantity of required test material is a unique feature that matches well with the small sample requirements using MS.

(2) Rapid mixing and processing—The liquid slugs generated within the microfluidic platform can be mixed rapidly into droplets due to short diffusion distances. Since the entire process can be carried out within a short time frame, the level of D/H back exchange and instability/removal of cofactors on proteins in HPLC running buffer can be greatly reduced.

(3) Constant and controllable environment—It is easy to control reaction parameters in the microfluidic environment and, as a result, the process is more stable than a similar reaction conducted in a macroscopic environment. Using microfluidics the increased surface area-to-volume ratio enhances of mass transport as a result of rapid diffusion, thus improving mixing and significantly improving heat transfer both into and out of the microfluidic reactor.

(4) Automated operations—The microfluidic chips described herein are digitally controlled and can be programmed to carry out step-by-step operations, thus greatly reducing human error and providing excellent reproducibility.

(5) Scalable for high-throughput—The microfluidic platform is scalable and can be operated in parallel to perform high-throughput HDX experiments by alternative or simultaneous injection into one or more ESI-MS.

(6) Modular and flexible—All of the microfluidic chip designs are designed for a stand-alone application so that they can be interconnected and programmed to execute complicated operations.

(7) Time interval—The time interval between generation of each droplet can be programmed so the microfluidic chip can generate droplets in parallel to process a desired number of droplets in a very short time period, such as seconds, to minimize the time difference in deuterium media incubation. It can also provide a unique distribution of droplets composed of proteins with various levels of deuteration, depending on their incubation time with D₂O.

The microfluidic devices described herein address the need to provide high-throughput methods for screening membrane protein stability. The thermal stability of soluble proteins correlates with the rate of global hydrogen exchange, which can be measured using HDX technologies. The Digital Microfluidic Droplet Generator (DMDG) Chip described herein are automated microfluidic platforms that can generate nanoliter droplets with defined compositions preprogrammed by the user. Because only very small amounts of test materials are required, it has been used to define optimal radiolabeling conditions of proteins and peptides and screen desired ratio combinations of individual building blocks for making self-assembled supramolecular nanoparticles (SNPs) with tailored biological properties. The utility of the DMDG chip was further extended by carrying out the HDX process. Illustrated herein are the multi-step HDX tests performed by a series of chip-based operations using the digital microfluidic droplet generator developed for controlled generation of droplets to be fed to the digital microfluidic platform.

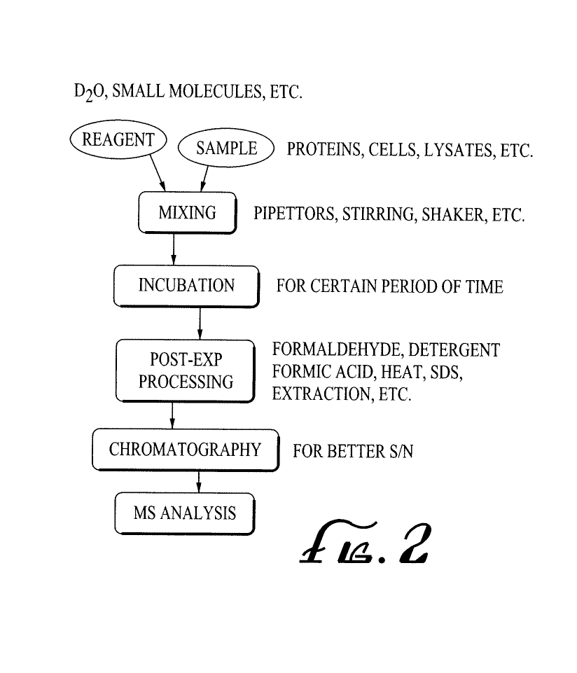

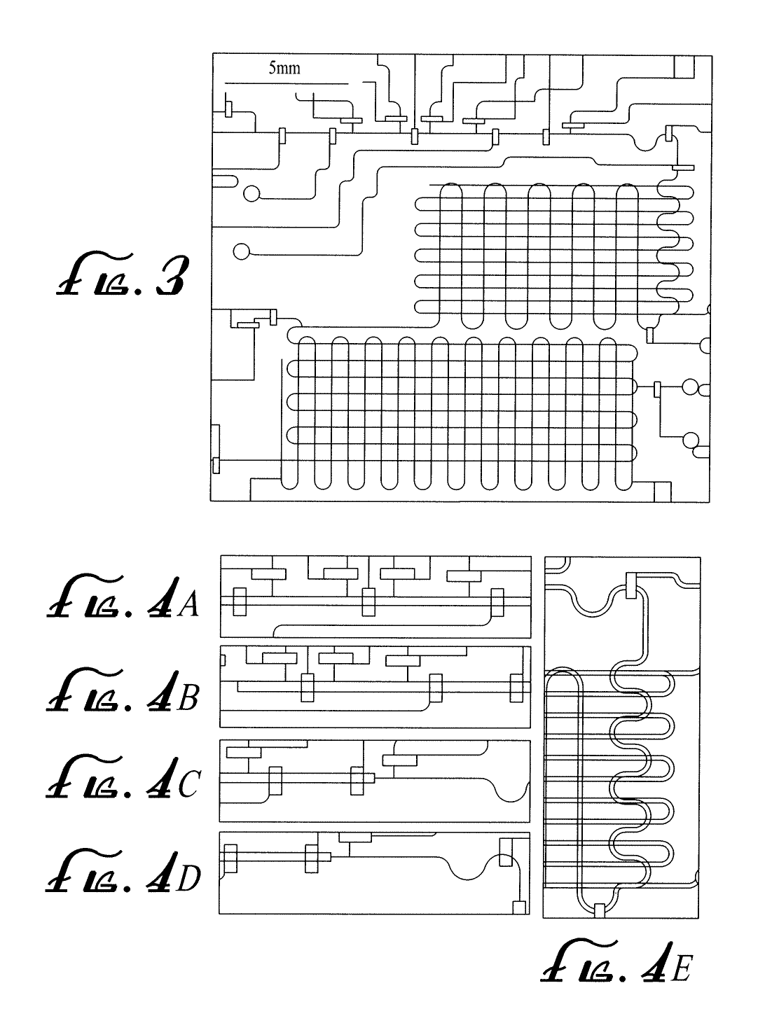

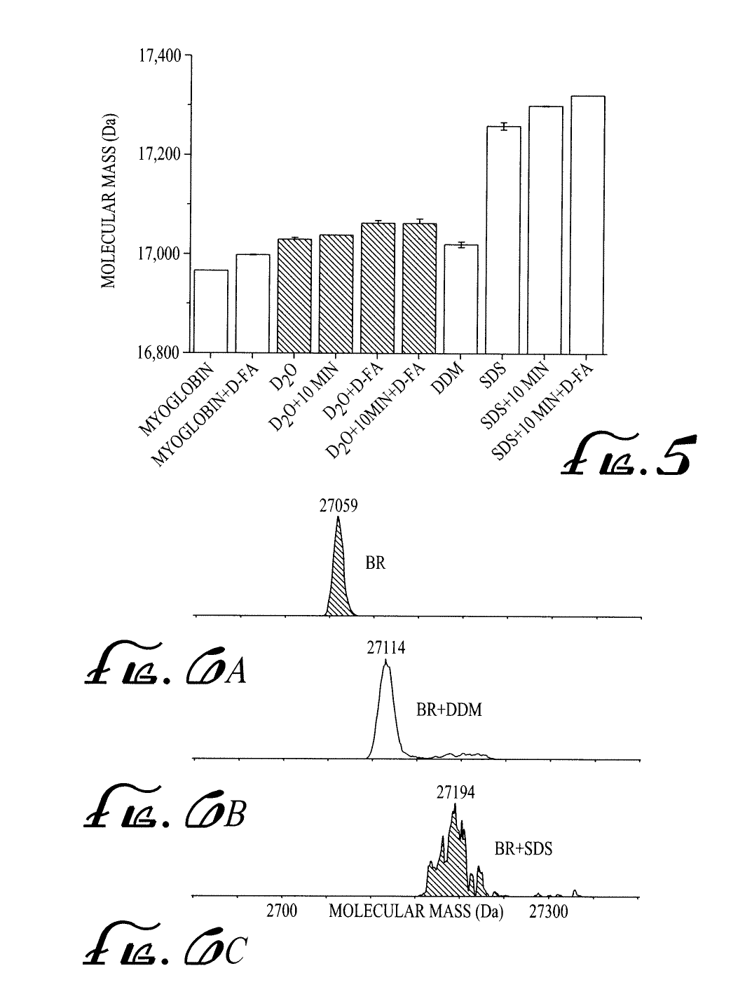

Each microdroplet can serve as an isolated miniature reactor for chemical and biological experiments with uniform and tunable volumetric ratio, temperature and composition. Digitized operations were developed for reliable generation of droplets from liquids, with control over their formation time, size, composition, and location in the channels of the chip. Unlike other approaches, the devices use integrated microvalves to control the formation of each droplet independent of most fluid properties, and they are capable of operating with very small volumes of samples or reagents. Furthermore, the droplet formation can be stopped and restarted on demand. The device architecture and operation is schematically shown in FIGS. 3-4a-e. FIGS. 3-4a-e are photographs of a two-layer PDMS microfluidic droplet generator chip shown divided into 5 components labeled as 4a-e. For illustration of functionality the control channels and microvalves can be filled with colored dyes, for example, red, yellow and blue food dyes can be used to visualize the filling chambers and reagent inlets. Each filling chamber is about 40 nL in volume. The droplet generator is coupled to a downstream serpentine channel to perform micromixing of droplets which then shows as green (a mixture of the yellow and blue dyes). The channels situated in the control layer (which can be filled with a different colored dye for illustration purposes, such as a pink food dye) can be used for cooling, heating, or applying vacuum to remove carrier gas between droplets.

Another unique aspect of the functionality of the droplet generator module and the microfluidic modules is the automation provided to manipulate fluids based on a trigger detecting the position of a liquid in the channels. Liquid-position sensing has been successfully integrated to provide reliable and repeatable control of fluid delivery to and between chambers and chips through the use of liquid detectors. For each delivery inlet, the system usually incorporates two liquid sensors placed along the tubing of the inlet line feeding the chip, a T-junction and vent channel and a microvalve. No on-chip sensors or special modifications to chip fabrication are necessary. The controller can automatically prime reagent inlets by manipulating the microvalve for venting such that the liquid sample arrives just short of the desired point in the chip at the desired time.

Hydrogen Deuterium Exchange (HDX)

Use of the mass spectrometry-based hydrogen/deuterium exchange (HDX-MS) can be used to determine protein structures by monitoring the rate and extent of deuterium exchange with backbone amide hydrogen using MS. The level of deuterium exchange depends on the solvent accessibility of the backbone amide hydrogen atoms and the conformation of the protein. As a sensitive analytical tool, MS can precisely measure the mass increase of final deuterated protein after deuterium exchange. It has been reported that the level of deuterium exchange can vary as much as 10⁸ times as a result of protein structure.

A typical HDX experiment begins by dilution of a protein solution at least about eight- to tenfold with a D₂O buffer. After each of a series of H/D exchange periods, the reaction is quenched by lowering the pH to about 2.3-2.5 (usually by addition of formic acid) and the temperature to about 0° C. In a top-down analysis, the sample is directly injected into an HPLC for separation and then into an ESI-MS for detection. In a bottom-up approach, the subsequent proteolysis is achieved by addition of a low-pH protease such as pepsin. A very rapid on-line high performance liquid chromatography (HPLC) step is used to prepare the sample for ESI-MS analysis. With an increasing H/D exchange period, the mass of each exposed segment of the protein increases, and this increase can be monitored by MS.

Since protein function is dictated by protein conformation, the relationship between isotopic exchange rates of main chain amide hydrogens in proteins and their secondary and tertiary structures can be used to distinguish their structures in solution. Therefore, HDX-MS has proven to be an extremely useful analytical method for the study of protein dynamics and changes to protein conformation.

Examples illustrating HDX-MS evaluations; first with myoglobin (Mb) and secondly with Bacteriorhodopsin (bR) using the DMDG chips are described herein.

DMDG Chip Setup and HDX Operation

A digital microfluidic droplet generator, in a two-layer polydimethylsiloxane (PDMS) microfluidic chip as shown in FIGS. 3 and 4a-e was used. The formation of the chip is described below. The upper layer of channels are for receiving the incoming reagents to generate droplets while the lower layer contains microvalve actuation channels. The core part of the micro-droplet generator consists of two microchannel segments isolated by valves forming two or more sealed, individual microchambers, each having a precise volume. This DMDG chip mixes protein with D₂O or/and detergent solution in very precise volume ratios. Valves are controlled by a LabView™ program through a control box (not shown) which allows pre-programing of the mixing ratios. Small-volumes (ca 40-60 nL) droplets can be generated one by one and the ratio of reagents can be changed in each droplet, if desired, during operation. Solutions are fed to the chip via separate inlets (see FIG. 4a). Their volumes can be varied by adjustment of microfluidic chamber sizes using microvalves. After the middle dividing valves open, two or three different reagents/samples are merged into a single end-to-end, multi-component droplet with a well-defined composition. The droplet is then ejected from the DMDG chip by delivering a stream of nitrogen; mixing within each droplet can be accomplished by flowing microdroplets along a microchannel (FIG. 4a). This sequence can be repeated multiple times; the droplets can be collected separately in a tip reservoir for MS analysis (FIGS. 4a & 4b). After collection of given number of droplets (from 50-400), concentrated formic acid solution was injected into the reservoir to quench/slow down the hydrogen/deuterium exchange process. The resultant solution was promptly loaded into the sample loop and injected into a HPLC and then the separated components were transferred to an ESI-MS. The rapid chromatographic separation of deuterated protein from detergent was accomplished using a short size-exclusion column. The time between completed collection of droplet to visualization of the corresponding protein signal appeared in MS is usually under 1 minute. All the HDX evaluations were carried out at room temperature and without cooling down any part of the device or apparatus below ambient temperature. The MS measurement is of the intact protein molecular weight. The HDX-MS data of two proteins in the presence of (i) non-denaturizing detergent [n-dodecyl β-D-maltoside (DDM)] (ii) denaturizing detergent [i.e. sodium dodecyl sulfate (SDS)] is shown in FIG. 5.

HDX MS Data of Myoglobin

Myoglobin (Mb) is an iron- and oxygen-binding protein found in the muscle tissue of almost all mammals. Mb is a soluble globular protein of 153 (or 154) amino acids, containing a heme prosthetic group in the center. The naïve horse myoglobin was measured with the hybrid linear ion trap/FTICR mass spectrometer (7 tesla, LTQ-FT Ultra, Thermo Scientific); its molecular mass was found to be 16,969.2 Da.

One of the biggest problems with the use of prior solution-phase HDX to study protein structure or dynamics is the back-exchange of proton for deuteron after the initial H/D exchange. According to literature, after quenching the HDX reaction, it is estimated that 30% of back-exchange happens during the chromatographic separation step, even if a very fast, cold (0-4° C.) HPLC is applied. Therefore, a first evaluation was designed to determine the extent of back-exchange using a DMDG chip.

### EXAMPLE 1

Myoglobin (Mb), Mb (3 mg/mL) and deuterated formic acid (D-FA) was mixed, with a volumetric ratio of 1:10, in the chip. A 24 Da increase in mass was observed which represents the extent of back-exchange during the quenching step. Next, HDX was first carried out by incubating Mb with D₂O and then quenched with FA, representing the full forward exchange. Then Mb was incubated with D₂O followed by quenching with D-FA, representing the full forward exchange without back-exchange during quenching. The observed mass shifts compared to pristine Mb were recorded as +46.3 Da for full forward exchange and +84.4 Da for full forward exchange without back exchange.

The above procedure demonstrated the successful utilization of chip-based HDX using on-line ESI-MS. The DMDG chip, which can easily interface with any type of mass spectrometer, has thus been shown to be an ideal technological platform for use of HDX-MS-based biological assay as a routine process.

### EXAMPLE 2

Next, we carried out a series of HDX experiments by incubating Mb with (i) D₂O (2:11, v/v), (ii) D₂O for 10 minutes (2:11, v/v), (iii) D₂O (2:11, v/v) then quenched with D-FA, and (iv) D₂O for 10 minutes (2:11, v/v) then quenched with D-FA, respectively.

The objective of those tests is to figure out to what extent of HDX can occur just by simply incubating Mb with D₂O. The observed mass shifts compared to pristine Mb were recorded as +46.3 Da for (i), +52.4 Da for (ii), and +84.4 Da for (iii) & (iv), respectively. It was concluded that:
(1) The final percentage of deuteration in Mb strongly depends on the amount of D₂O and D-FA in the final solution, i.e. % of deuterium;
(2) The extent of final deuteration of Mb also correlates to incubation time. 10-minute incubation in D₂O gave higher % of deuterium incorporation within Mb. However, this effect seems to be diminished while using D-FA quenching. It is possible that the fast forward exchange in D-FA filled in deuterium around all the possible sites; and
(3) The percentage of back-exchange is a bit higher than the estimated value reported in literature [i.e. 25.3 Da if calculated using 30% of maximal HDX (84.4 Da)]. Direct comparison between (i) & (iii) and (ii) & (vi) suggested that the back-exchange might be 38.1 Da in the case of 0 minute incubation and 32 Da for 10 minute incubation. This may be the result of running the evaluation at room-temperature.

### EXAMPLE 3

Tests were then performed to investigate the effect of different detergents. HDX experiments were conducted using Mb with (v) 1% SDS in D₂O and D₂O (2:1:10, v/v/v), (vi) 1% SDS in D₂O and D₂O for 10 minutes (2:1:10, v/v/v), and (vii) 1% DDM in D₂O and D₂O (2:1:10, v/v/v), respectively. The molecular mass differences between the final deuterated protein and Mb were +292.2 Da for (v), +332.5 Da for (vi), and +53.1 Da for (vii), respectively. It was concluded that
(1) There is a significant difference between non-denaturing detergent (i.e. DDM) and denaturing detergent (i.e. SDS). The % of deuteration was very different from that obtained using D₂O;
(2) Longer incubation time with 1% SDS in D₂O was again correlated with higher final molecular mass. The percentage of HDX in Mb increased when using “denaturing” SDS vs. “non-denaturing” DDM; and
(3) If the back-exchange was estimated using the data obtained from D₂O HDX experiment (about 32 Da), the % of forward-exchange of Mb with 1% SDS in D₂O is very similar to that of the maximal HDX with D₂O alone.

The data showed distinguishable discrimination between the two detergents, “denaturing” SDS and “non-denaturing” DDM, in the case of Mb. Nevertheless, the evaluations have demonstrated the very controllable use of HDX using DMD. Detailed mass data and experimental conditions are summarized in Table 1. The detailed information of each Mb HDX experiment, (sample no. 1-9), such as volumetric ratios of protein, D₂O, and detergent as well as incubation time, are listed in Table 1.

HDX MS Data of Bacteriorhodopsin

Bacteriorhodopsin (bR) is a protein used by Archaea, most notably Halobacteria. It acts as a proton pump by capturing light energy; it is then used to move protons across the membrane out of the cell. The resulting proton gradient is subsequently converted into chemical energy. bR typical comprises many integral membrane proteins and has less ionizable side-chain functionalities. As a result, it is relatively poorly charged in ESI-MS compared with soluble proteins. bR was applied as a test case to demonstrate that the on-chip HDX MS analysis can also be performed with membrane proteins solubilized in a non-denaturing detergent, such as 3[(3-cholamidopropyl)-dimethylammonio]-1-propanesulfonate (CHAPS).

Size-exclusion chromatography was used to separate the resulting deuterated bR from small-molecule-based detergents and contaminants including CHAPS and lipids. The eluting buffer (developed in Whitelegge's lab) contained chloroform, methanol, 1% formic acid in H₂O (4:4:1, v/v/v), which is an ideal solution providing delicate balance to dissolve both aqueous components, hydrophobic lipids and membrane proteins.

Our measurements showed that the molecular mass of bR is 27059 Da. An HDX experiment was first conducted on bR with detergents to estimate the level of back-exchange with D-FA. The mass differences of back-exchange was 8 Da using DDM and 24 Da using SDS, respectively. The effect of detergent addition was also accessed and it was found that there was a 55 Da molecular weight increase in the case of DDM and 135 Da increase in the case of SDS. The results (Table 1, experiments 10-14) show the volumetric ratios of protein D₂O, detergent and incubation times and clearly demonstrates the extent of HDX of bR is dominated by the nature of the detergent. With the denaturing SDS, the back-exchange is higher than that of non-denaturing DDM (24 vs. 8 Da). The forward-exchange was also found to be higher when using SDS compared to DDM (135 vs. 55 Da). FIG. 6 is a graph comparing the HDX-MS analyses of molecular mass for Bacteriorhodopsin as received (A), with DDM in D₂O then quenched with FA (B) and with SDS in D₂O then quenched with FA (C).

Previous studies showed the retinal chromophore of bR is susceptible to hydrolysis under the acidic conditions. However, we saw very little, if any, of the detachment of heme from bR. It is believed that this can be due to the rapid on-chip processing and swift size-exclusion HPLC which prevented decomposition. Detailed mass data and experimental conditions are summarized in Table 1.

TABLE 1

| Sample | Molecular | | |
|---|---|---|---|
| No. | Mass (Da) | SD | Description |
| 1 | 16967.17 | 0.01 | Myoglobin (Mb)^{a} |
| 2 | 16998.87 | 3.62 | Mb + D-FA |
| 3 | 17030.40 | 1.30 | Mb + D₂O |
| 4 | 17039.12 | 4.82 | Mb + D₂O + 10 min |
| 5 | 17063.45 | 9.06 | Mb + D₂O + D-FA^{b} |
| 6 | 17062.67 | 0.72 | Mb + D₂O + 10 min + D-FA |
| 7 | 17259.40 | 5.22 | Mb + SDS^{c} |
| 8 | 17299.72 | 7.12 | Mb + SDS + 10 min |
| 9 | 17321.38 | 1.44 | Mb + SDS + 10 min + DFA |
| 10 | 17020.34 | 0.69 | Mb + DDM^{d} |
| 11 | 27059 | n.a. | Bacteriorhodopsin (bR)^{f} |
| 12 | 27114 | n.a | bR + DDM^{g} |
| 13 | 27067 | n.a. | bR + DDM in H₂O + D-FA^{g} |
| 14 | 27194 | n.a. | bR + SDS^{g} |
| 15 | 27083 | n.a | bR + SDS in H₂O + D-FA^{g} |

^{a}pure Mb (<1 mg/mL in 10 mM HEPES, pH 7.4), 2 μL for total injection;
^{b}Mb: deuterated formic acid (D-FA) = 1:10;
^{c}Mb: D₂O = 2:11;
^{d}Mb: 1% SDS in D₂O: D₂O = 2:1:10;
^{e}Mb and 1% DDM in D₂O: D₂O = 2:1:10.
In samples 1-9 the final solutions were quenched by either FA or D-FA (sample: FA = 1:10);
^{f}pure bR (ca 5.5 mg/mL), 2 μL for total injection;
^{g}bR: detergent: FA or D-FA = 8:60:24.

Table 1 presents data from the HDX-MS analyses of myoglobin (Mb) and bacteriorhodopsin (bR).

### EXAMPLE 4

An aqueous-organic micro-size-exclusion chromatography system (micro-SEC) compatible with electrospray-ionization mass spectrometry was developed which allows rapid separation of polypeptides from salts, detergents and formic acid. A mobile phase of chloroform/methanol/1% aqueous formic acid (4/4/1; v/v) was coupled with a modified silica matrix packed into various column geometries. The benchmark 4.6×300 mm format was compared with custom 2.0×150 and 1.0×50 mm columns. Samples purified in this way were analyzed using online electrospray-ionization mass spectrometry with low- or high-resolution mass analyzers. However, they can also be suitably evaluated using static nanospray analysis. Top-down data was analyzed using a commercial algorithm (ProsightPC™) and custom proteome databases.

A number of different configurations were investigated using myoglobin and bacteriorhodopsin as standards. By proper selection of loop volume, column geometry and flow rate, protein mass spectra can be obtained within 40 seconds and columns can be regenerated in less than 5 minutes for protein loads in the sub-microgram range for both standards using the 1 mm column. One immediate advantage of the rapidity of the separation relates to preservation of labile post-translational modifications. The model integral membrane protein bacteriorhodopsin has a retinal chromophore that becomes susceptible to hydrolysis under semi-denaturing conditions such that around 50% is lost after the 8-10 minutes that it takes to elute the protein from the classic 4.6×300 mm column. However, with the 40 second micro-SEC method practically no hydrolysis of the chromophore was observed. There are tremendous throughput benefits which allows collection of 12 or more samples per hour using micro-SEC compared to prior techniques that provided only about 2 samples/hour.

The micro-SEC system described is proving suitable for top-down mass spectrometry of 50 kDa polytopic integral membrane proteins including prokaryotic chloride channels and mammalian beta-adrenergic receptors. In this context, another advantage relates to the relatively weak ion currents that tend to be observed for larger membrane proteins, demanding high peak protein concentrations. Reduction in loop volume from 100 to 5 microLitre with matching miniaturization of column geometry allows a twenty fold increase in peak protein concentration for a limited sample mass. For samples where fraction collection is undesirable peak parking conditions have been established for extended averaging of transients essential for robust top-down high-resolution mass spectrometry of demanding targets.

A novel aspect of electrospray-ionization mass spectrometry of nanogram samples of intact integral membrane proteins is that it allows full proteome coverage top-down analyses while significantly reducing or eliminating sample degradation due to the much longer processing time required by past techniques.

DMDG Chip Fabrication

Microfluidic chips were fabricated using polydimethylsiloxane (PDMS) substrates and standard multi-layer soft lithography technology. Two different sets of molds (a fluidic layer mold and a control layer molds) were separately fabricated by standard photolithographic process. For a standard chip, the fluidic layer mold was patterned with 45 μm thick positive photoresist (AZ 50XT) on the silicon wafer to provide a channel width of 300 μm and a channel height of 45 μm. The control layer mold was patterned with a negative photoresist (SU8-2050) on the silicon wafer to provide a channel width of 100 μm and a channel height of 40 μm. In order to achieve reliable performance of the valves, the control channel mold has 250 μm widths in portions where the valve modules are to be located. Before pouring PDMS prepolymer on them, all molds were pretreated by exposure to trimethylsilyl chloride (TMSC1) vapor for 10 minutes. Well-mixed PDMS prepolymer (GE RTV615, total 36 g, mixing ratio A:B=5:1) was poured onto the fluidic layer molds to provide a 6 mm-thick fluidic layer. Another portion of PDMS prepolymer (GE RTV615, total 10 g, mixing ratio A:B=20:1) was mixed and then spin-coated onto the control layer molds at 1500 RPM for 60 sec. The fluidic and control layers were cured in an 80° C. oven for 15 minutes and 18 minutes, respectively. After curing, the fluidic layers were peeled from the mold, aligned onto the corresponding control layers, and then baked at 80° C. for at least 6 hours so that they become bonded together. The assembled and bonded layers were then peeled off of the control layer molds. Holes were then punched to form ports connected to the fluidic layer channels for reagent inlets and outlets, and ports were connected to the control layer channels for valve actuation with hydraulic fluid. Adhesion of the layers to clean glass microscope slides to seal the control channels was achieved by oxygen plasma bonding. The assembled microfluidic device was baked in an oven at 80° C. for an additional 72 hours to restore the intrinsic hydrophobicity of PDMS surfaces needed to minimize the residue lost on channel walls when moving aqueous slugs of materials fed through the microchannels.

Chip Control Interface

The pneumatic control system consists of 2 sets of 48-channel manifolds (electronic solenoid valves, SMC Series S070 or the like). On-chip microvalves were actuated by pressurizing the corresponding control channel, filled with water, to 60 psi. When the manifold is activated, pressure is transferred from the solenoids to the chip via PTFE microbore tubing (0.022″ inner diameter, Cole Parmer) connected to 22-gage stainless steel tube. The valves in the control layer then close the corresponding fluidic channel. All the valves are automatically controlled through a control system by a software program written in LabView™ (National Instruments).

Setup of HDX Evaluations

The DMDG chip is composed of three functional parts (FIG. 4a): (1) a droplet generation core (long channel in the middle, connected to vacuum lines on both ends), where specific quantities of reagents are measured and merged into composition-specific droplets; (2) a peristaltic pump, of which is connected to N₂, produces serial compressed nitrogen pulses that can precisely deliver intact droplets to the desired location and (3) a mixing channel (the serpentine channel in the middle of the chip, directly connected to the chip outlet).

Protein solution, detergent, H₂O and D₂O are first loaded into small glass vials and transferred under positive N₂ pressure via PTFE microbore lines directly into the corresponding inlets on the DMDG chip (see 4D). After all the lines are primed, the DMDG chip was washed with H₂O (3-5 μL) and D₂O (1-3 μL) and then dried with a stream of N₂ before each use. Microvalves were used to isolate each reagent or sample inlet so that the incoming reagents are not in contact with the sample to be evaluated until the moment of droplet formation. Once combined in a channel they will rapidly mix and react while moving along the microfluidic channel.

The outlet of DMDG chip is connected with a reservoir for product collection. For initial testing a 1-mL disposable pipette tip was used. As droplets came out of the microbore tube, they sprayed out and stuck onto the pipette tip due to surface tension. At the end of a procedure, a fixed volume of formic acid (about 24 μL) was delivered to the same tip. Finally the entire solution was mixed thoroughly and manually loaded into the sample loop using a syringe via the waste line connected to the HPLC injector.

The rapid HPLC separation was carried out at 50 μL min with a custom size exclusion column (1mm×5cm, filled with Tosoh TSK-gel SW2000) using an elution buffer containing chloroform, methanol, and 1% formic acid in H₂O (4:4:1, v/v/v). The eluent was directed to the electrospray ionization source of an ESI-MS. The mass spectra were record on either a 7T Thermo Scientific LTQ-FT Ultra hybrid linear ion trap/FTICR mass spectrometer or a Pekin Elmer Sciex API III triplet quadrupole mass spectrometer fitted with Inospray source. The mass spectrometer was tuned and calibrated before the HDX experiment by flow injection of a mixture of polyproylene glycol (PPG). The first peak containing a fraction of deuterated protein generally showed up around 30-40 seconds after injection. FTICR spectra were processed using ProSightPC™ (Thermo Scientific) to produce a monoisotopic mass.

1. A method of providing separation, analysis and characterization of membrane proteins comprising:
a. preparing a solution comprising the membrane proteins in a carrier solution containing a solubilizing detergent,
b. using a digital microfluidic droplet generator, feeding micro-droplets of the solution into a microfluidic processing platform for incubation or reaction,
c. feeding the incubated or reacted droplets from a platform outlet to an HPLC column to separate the incubated or reacted proteins, and
d. feeding the separated proteins into a mass spectrometer for analysis thereof.

2. The method of claim 1 where hydrogen deuterium exchange is performed on the micro-droplets of proteins in solution within the confines of microchannels in the microfluidic processing platform.

3. The method of claim 1 wherein the digital microfluidic droplet generator and the microfluidic processing platform constitute a single integral chip.

4. The method of claim 1 configured for detecting protein-ligand interaction and determining a dissociation constant K_{d}.

5. The method of claim 1 wherein a protein mass spectra analysis is obtained within about 40 seconds of receiving separated proteins from the HPLC column.

6. The method of claim 1 wherein the HPLC column is regenerated within about 5 minutes.

7. An assembly for separation, analysis and characterization of membrane proteins comprising:
a. delivery apparatus for providing one or more delivered materials comprising carrier solutions, solubilizing detergent, reactants and membrane proteins for subsequent mixing and processing,
b. a mixing apparatus for combining the delivery materials,
c. a digital microfluidic droplet generator for receiving and combining said delivered materials, forming micro-droplets of said combined delivered materials and isolating and feeding micro-droplets of a solution of the combined delivered materials into microchannels in a microfluidic processing platform, the micro-droplets of proteins delivered materials in solution being processed within the microchannels in the microfluidic processing platform,
d. an HPLC apparatus configured to receive and separate the processed delivered materials exiting the microfluidic processing platform and separate proteins, and
e. a mass spectrometer for analysis of the separated proteins exiting the HPLC apparatus, of said assembly programmed to automatically and repetitively control processing conditions, chemical reactions and processes conducted within the microchannels of the chip.

8. The assembly of claim 7 wherein the digital microfluidic droplet generator and the microfluidic processing platform constitute a single integral chip.

9. The assembly of claim 7 configured for detecting protein-ligand interaction and determining a dissociation constant K_{d}.

10. The assembly of claim 7 configured for detecting changes in protein confirmation utilizing HDX-MS techniques.

11. The assembly of claim 7 configured for subjecting proteins therein to selected amounts of one or more of deuteration and denaturation for protein structural analysis.

12. The assembly of claim 7 wherein a protein mass spectra analysis is obtained within about 40 seconds of delivery of a sample from the HPLC apparatus.

13. The assembly of claim 7 wherein an HPLC column in the HPLC apparatus is regenerated within about 5 minutes.

14. The apparatus of claim 7 wherein the delivered materials are combined in the digital microfluidic droplet generator.
